SEQUENCE LISTING (18 sequences)

SEQ ID NO: 1 - DNA, 20 bp, Artificial Sequence
  Forward primer sequence
aacctgcatcttccccaagt

SEQ ID NO: 2 - DNA, 20 bp, Artificial Sequence
  Reverse primer sequence
ccttttaccaacccaacacg

SEQ ID NO: 3 - DNA, 24 bp, Artificial Sequence
  Forward primer sequence
tgcaaatggttattgattgctctc

SEQ ID NO: 4 - DNA, 24 bp, Artificial Sequence
  Reverse primer sequence
atttgtttcccatgacctgaaaga

SEQ ID NO: 5 - DNA, 24 bp, Artificial Sequence
  Forward primer sequence
tcatatttatgctttccttgccgt

SEQ ID NO: 6 - DNA, 24 bp, Artificial Sequence
  Reverse primer sequence
gtgatcctctcctaaggacttcca

SEQ ID NO: 7 - DNA, 21 bp, Artificial Sequence
  Forward primer sequence
ctaacctgcatcttccccaag

SEQ ID NO: 8 - DNA, 21 bp, Artificial Sequence
  Reverse primer sequence
tttaccaacccaacacgacac

SEQ ID NO: 9 - DNA, 21 bp, Artificial Sequence
  Forward primer sequence
ccagaggtatgaattctgtgt

SEQ ID NO: 10 - DNA, 21 bp, Artificial Sequence
  Reverse primer sequence
cttgcaaattgttgtaatgaa

SEQ ID NO: 11 - DNA, 21 bp, Artificial Sequence
  Forward primer sequence
aacgaagcaagcaaaacaatc

SEQ ID NO: 12 - DNA, 20 bp, Artificial Sequence
  Reverse primer sequence
accccaccacttcatctctc

SEQ ID NO: 13 - DNA, 24 bp, Artificial Sequence
  Forward primer sequence
aagtcagatcatttcagattaaca

SEQ ID NO: 14 - DNA, 24 bp, Artificial Sequence
  Reverse primer sequence
cgaattgacgataaatacaaaata

SEQ ID NO: 15 - DNA, 24 bp, Artificial Sequence
  Forward primer sequence
gaaattcaaattttaaagaagtcc

SEQ ID NO: 16 - DNA, 23 bp, Artificial Sequence
  Reverse primer sequence
ttgtatagtacttcaaggccact

SEQ ID NO: 17 - DNA, 24 bp, Artificial Sequence
  Forward primer sequence
gatacttatgcaattaggacacaa

SEQ ID NO: 18 - DNA, 20 bp, Artificial Sequence
  Reverse primer sequence
gtaacaatggtggaaggaag

(19) United States
(12) Plant Patent Application Publication
(10) Pub. No.: US 2020/0323117 P1
(43) Pub. Date: Oct. 8, 2020

(54) Cornus florida Tree Named 'Erica's Appalachian Sunrise'

(50) ;

(71) Applicant: UNIVERSITY OF TENNESSEE RESEARCH FOUNDATON, KNOXVILLE, TN (US)

(72) Inventors: Robert N. Trigiano, Knoxville, TN (US); Phillip A. Wadl, Charleston, SC (US)

(21) Appl. No.: 16/602,052

(22) Filed: Jul. 26, 2019

Related U.S. Application Data
(60) Provisional application No. 62/830,694, filed on Apr. 8, 2019.

(51) Int. Cl. A01H 6/00

(52) U.S. Cl. PLT/220

(57) ABSTRACT

A new and distinct cultivar of flowering dogwood tree, which produces both fully dark red bracts and lighter red to pink bracts is provided. This dogwood tree is botanically known as Cornus florida and referred to by the following cultivar name: ‘Erica's Appalachian Sunrise’.

### CROSS-REFERENCE TO A RELATED APPLICATION

This application claims the benefit of U.S. Provisional Application Ser. No. 62/830,694, filed Apr. 8, 2019, the disclosure of which is hereby incorporated by reference in its entirety, including all figures, tables and drawings.

The Sequence Listing for this application is labeled “Seq-List.txt” which was created on Oct. 29, 2019 and is 4 KB. The entire content of the sequence listing is incorporated herein by reference in its entirety.

This invention was made with Government support under Contract No. NACA-58-6062-6 awarded by the U.S. Department of Agriculture. The Government has certain rights in the invention.

### BACKGROUND OF THE INVENTION

The present invention relates to a new and distinct cultivar of flowering dogwood tree with a mixture of both fully dark red and lighter red bracts. This new cultivar was the result of a controlled cross that produced a few seeds, which were planted in a greenhouse in Knoxville, Tenn. This new cultivar was discovered among the resulting seedlings. This dogwood tree is botanically known as Cornus florida L. and is hereinafter referred to by the following cultivar name: ‘Erica's Appalachian Sunrise’. Analysis has shown that this new dogwood cultivar is the result of self-pollination of the dogwood cultivar ‘Cherokee Brave’ (U.S. Plant Pat. No. 10,166). The seedling of ‘Erica's Appalachian Sunrise’ was harvested on its own rootstock. Results have shown that the unique features of this new dogwood cultivar are stable and reproduced true-to-type in successive vegetative generations.

### BRIEF DESCRIPTION OF THE DRAWINGS

FIG. 1. Photograph of one type of bracts and flowers of the dogwood cultivar ‘Erica's Appalachian Sunrise’. This bract has fully dark red coloration and is less than about 5% of the bracts produced by the cultivar. Colors in the photograph may differ from actual colors due to lighting and light reflectance.

FIG. 2. Photograph of ‘Erica's Appalachian Sunrise’ dogwood cultivar showing the other type of bract and flowers produced more than about 95% of the time on the dogwood tree, which has lighter red or more pink bracts. Colors in the photograph may differ from actual colors due to lighting and light reflectance.

FIG. 3. Photograph of new leaf growth on ‘Erica's Appalachian Sunrise’. Colors in the photograph may differ from actual colors due to lighting and light reflectance.

FIG. 4. Photographs of the bracts and flowers of dogwood cultivars ‘Cherokee Brave’ and ‘Karen's Appalachian Blush,’ (U.S. Plant Pat. No. 13,165), which were initially crossed. Results show that the resulting F1 cultivar, ‘Erica's Appalachian Sunrise’, is not, as was expected, related to ‘Karen's Appalachian Blush’, but is the result of self-pollination of ‘Cherokee Brave’ (U.S. Plant Pat. No. 10,166). It can be seen that the lighter red or pink bracts of ‘Erica's Appalachian Sunrise’ (shown in FIG. 2) closely resemble the bracts of the parent, ‘Cherokee Brave’.

FIG. 5. Photograph of the less commonly produced bracts and flowers, less than about 5%, of the dogwood cultivar ‘Erica's Appalachian Sunrise’ (top—fully dark, red bracts), and those of ‘Karen's Appalachian Blush’ (bottom left) and ‘Cherokee Brave’ (bottom right).

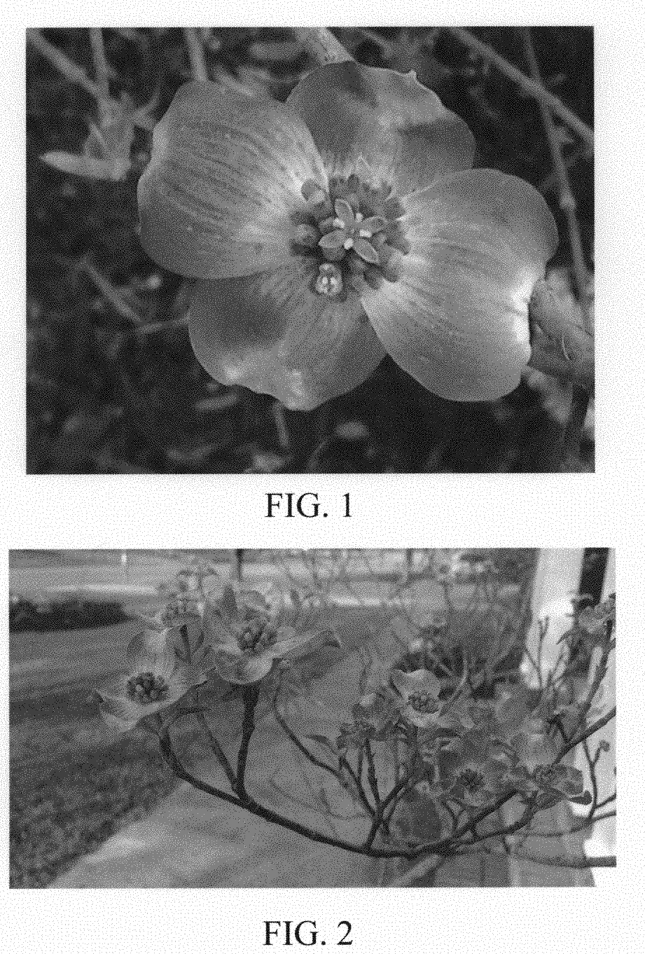

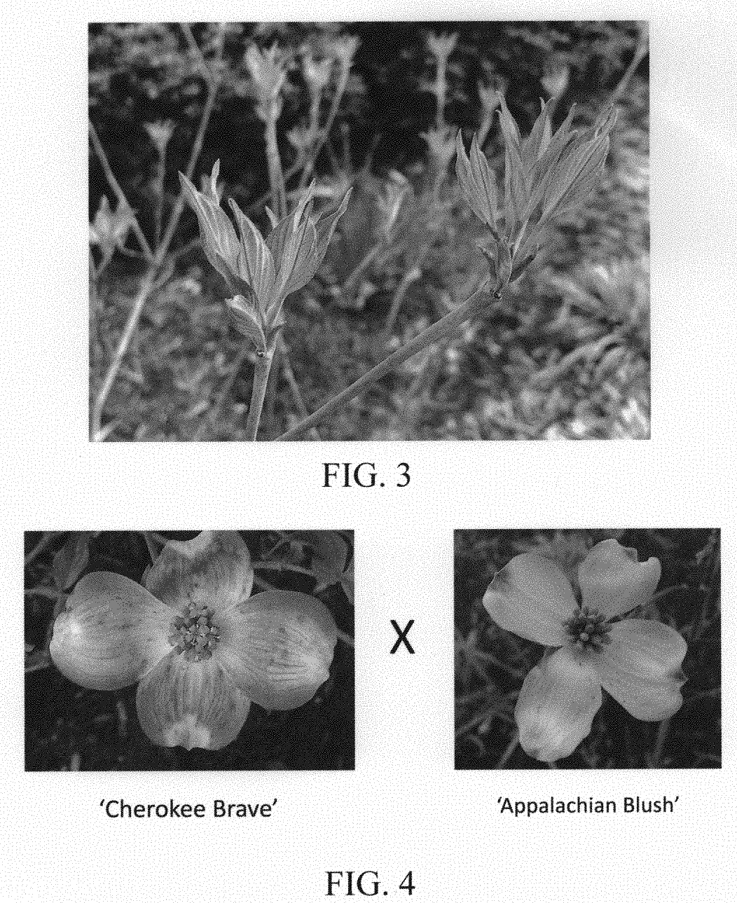

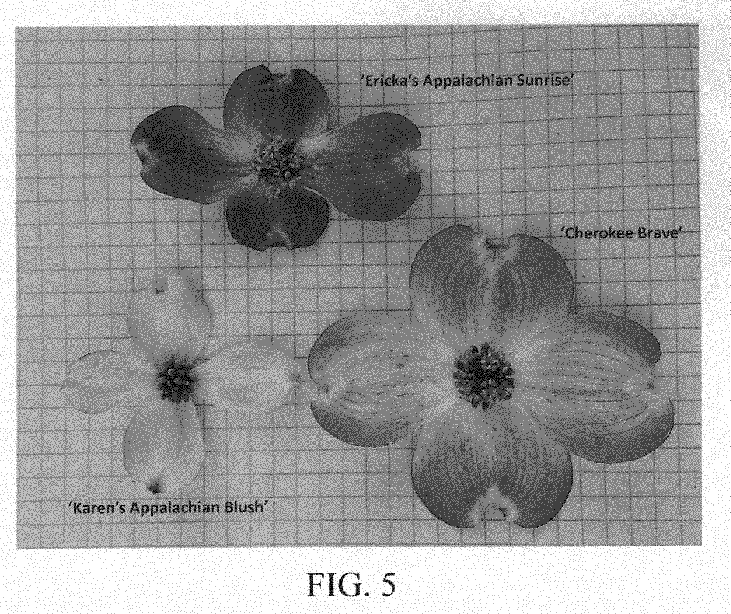

### DETAILED DESCRIPTION OF THE NEW VARIETY

A new and distinct cultivar of flowering dogwood tree producing both fully dark red bracts and lighter red or pink bracts. Both types of bracts are significantly smaller than the bracts of the parent cultivar, ‘Cherokee Brave’. This dogwood tree cultivar is botanically known as Cornus florida and referred to by the following cultivar name: ‘Erica's Appalachian Sunrise’. This cultivar appears to be highly resistant to powdery mildew caused by Erisphe pulchra.

This new and distinct dogwood tree cultivar is a product of self-pollination of the dogwood cultivar ‘Cherokee Brave’. The subject dogwood tree cultivar differs from ‘Cherokee Brave’ in that the instant cultivar produces significantly smaller and both fully dark red bracts and lighter red bracts and also exhibits greater resistance to powdery mildew.

### DETAILED BOTANICAL DESCRIPTION

The following observations, measurements and comparisons describe this cultivar grown in Maryville, Tenn. Trees used for this description were about ten (10) years old. Both the fully dark red bracts and lighter red to pink bracts are substantially the same size, though significantly smaller than the bracts produced by the parent cultivar. Plant hardiness is expected to be zones 4-9. The color characteristic descriptions use color references to The Royal Horticultural Society (R.H.S.) Colour Chart, The Royal Horticultural Society, London, UK, 4^{th} Edition, 2001, except where general terms of ordinary dictionary significance are used.

A bee-mediated pollination between the dogwood cultivars ‘Cherokee Brave’ and ‘Karen's Appalachian Blush’ was conducted in April of 2009. Seeds were collected from both cultivars and planted in a greenhouse in Knoxville, Tenn. This new and distinct dogwood tree cultivar was discovered among the planting and germination of the seeds harvested from ‘Cherokee Brave’. The following Table 1 shows the alleles at nine (9) loci compared between the cultivars ‘Karen's Appalachian Blush’ (U.S. Plant Pat. No. 13,165), ‘Cherokee Brave’, and the new cultivar ‘Erica's Appalachian Sunrise’. As seen in Table 1, the alleles for ‘Erica's Appalachian Sunrise’ are identical at all nine (9) loci to those of ‘Cherokee Brave’ and have no alleles that match ‘Karen's Appalachian Blush’. This demonstrates conclusively that dogwood cultivar ‘Erica's Appalachian Sunrise’ was the result of self-pollination of ‘Cherokee Brave’. Asexual reproduction of ‘Erica's Appalachian Sunrise’ by grafting of axillary buds onto seedling rootstocks has shown that the unique features of this new dogwood cultivar are stable and reproduced true-to-type in successive vegetatively propagated generations.

TABLE 1
Allelic Comparisons at Nine (9) loci for dogwood cultivars ‘Karen's Appalachian
Blush’ (KAB), ‘Cherokee Brave’ (CB), and ‘Erica's Appalachian Sunrise’ (EAS)

| | Loci/Primer | | | | | | | | |
|---|---|---|---|---|---|---|---|---|---|
| Cultivar | CF213 | CF191 | CF273 | CF322 | CF585 | CF597 | CF634 | CF713 | CF562 |
| KAB | 270:270 | 132:169 | 140:144 | 137:173 | 167:187 | 114:126 | 120:126 | 154:154 | 208:208 |
| (as base- | | | | | | | | | |
| pair size) | | | | | | | | | |
| CB | 267:278 | 144:144 | 133:142 | 154:154 | 174:174 | 105:120 | 113:113 | 144:144 | 212:225 |
| (female) | | | | | | | | | |
| (as base- | | | | | | | | | |
| pair size) | | | | | | | | | |
| EAS | 267:278 | 144:144 | 133:142 | 154:154 | 174:174 | 105:120 | 113:113 | 144:144 | 212:225 |
| (F1) | | | | | | | | | |
| (as base- | | | | | | | | | |
| pair size) | | | | | | | | | |

TABLE 2
Simple Sequence Repeats and Associated Primers
for Nine Loci shared by the Dogwood Cultivars
‘Erica's Appalachian Sunrise’ and ‘Cherokee Brave’

| GenBank | | Forward and | Re- |
|---|---|---|---|
| acces- | | Reverse Primer | peated |
| sion no. | Locus | Sequences (5′-3′) | Seq. |
| ED651856 | CF191 | F: AACCTGCATCTTCCCCAAGT | (AG)₂₀ |
| | | (SEQ ID NO: 1) | T(GA)₁₂ |
| | | R: CCTTTTACCAACCCAACACG | (GAA)₄ |
| | | (SEQ ID NO: 2) | |
| ED651874 | CF213 | F: TGCAAATGGTTATTGATTGCT | (CT)₉ |
| | | CTC (SEQ ID NO: 3) | (GT)₁₂ |
| | | R: ATTTGTTTCCCATGACCTGAA | |
| | | AGA (SEQ ID NO: 4) | |
| ED651920 | CF273 | F: TCATATTTATGCTTTCCTTGC | (AC)₁₄ |
| | | CGT (SEQ ID NO: 5) | |
| | | R: GTGATCCTCTCCTAAGGACTT | |
| | | CCA (SEQ ID NO: 6) | |
| ED651957 | CF322 | F: CTAACCTGCATCTTCCCCAAG | (AG)₂₀ |
| | | (SEQ ID NO: 7) | TG(AG)₁₂ |
| | | R: TTTACCAACCCAACACGACAC | |
| | | (SEQ ID NO: 8) | |
| ER870584 | CF562 | F: CCAGAGGTATGAATTCTGTGT | (GT)₁₆ |
| | | (SEQ ID NO: 9) | |
| | | R: CTTGCAAATTGTTGTAATGAA | |
| | | (SEQ ID NO: 10) | |
| ER870607 | CF585 | F: AACGAAGCAAGCAAAACAATC | (AT)₇ |
| | | (SEQ ID NO: 11) | (GT)₁₁ |
| | | R: ACCCCACCACTTCATCTCTC | |
| | | (SEQ ID NO: 12) | |
| ER870619 | CF597 | F: AAGTCAGATCATTTCAGATTA | (AC)₁₃ |
| | | ACA (SEQ ID NO: 13) | |
| | | R: CGAATTGACGATAAATACAAA | |
| | | ATA (SEQ ID NO: 14) | |
| ER870656 | CF634 | F: GAAATTCAAATTTTAAAGAAG | (AG)₁₄ |
| | | TCC (SEQ ID NO: 15) | |
| | | R: TTGTATAGTACTTCAAGGCCA | |
| | | CT (SEQ ID NO: 16) | |
| ER870735 | CF713 | F: GATACTTATGCAATTAGGACA | (TC)₁₈ |
| | | CAA (SEQ ID NO: 17) | |
| | | R: GTAACAATGGTGGAAGGAAG | |
| | | (SEQ ID NO: 18) | |

The cultivar ‘Erica's Appalachian Sunrise’ has some phenotypic similarities to the cultivar ‘Cherokee Brave’, but also distinct differences. The following Table 3 provides a comparison of those characteristics for each cultivar that have been observed. Measurements are provided as averages (with ranges also provided as indicated):

TABLE 3
Comparison of Characteristics for Three Dogwood Tree Cultivars

| | | ‘Erica's | | | | ‘Karen | |
|---|---|---|---|---|---|---|---|
| | | Appalachian | | ‘Cherokee | | Appalachian | |
| | Character | Sunrise | | Brave’ | | Blush’ | |
| 1 | Tree Height | 2 | meters | 2-3 | meters | 2-3 | meters |
| | (observation) | (at 8 years) | | (10 -15 years) | | (10 -15 years) | |
| 2 | Tree Form | Branching/Spreading | | Branching/Spreading | | Narrow few | |
| | | | | | | branches | |
| 3 | Growth Rate | Slow 16 cm/year | | Moderate 24 cm/year | | Slow 12 cm/year | |
| 4 | Spread of Tree | 1.5 | meters | 2.0 | meters | 0.8 | meters |
| 5 | Trunk Diam. at | 6.5 | cm | 8 | cm | 5 | cm |
| | 1 meter | | | | | | |
| 6 | Trunk Texture | Smooth | | Smooth | | Smooth | |
| 7 | Primary Trunk | 144A New Growth | | 144A New Growth | | 144C New Growth | |
| | Color/New | Older Mature | | Older Mature | | Older Mature | |
| | Branches/ | 201B/196B | | 201B/196B | | 202A/196B | |
| | Texture | Smooth | | Smooth | | Smooth | |
| 8 | Presence of | Red with Green | | Red with Green | | Green 143B | |
| | anthocyanin | Mainly 61B with | | Mainly 61B with | | | |
| | (observation) | some 143C | | some 143C | | | |
| | Coloration by | | | | | | |
| | anthocyanin on | | | | | | |
| | the immature leaf | | | | | | |
| | upper side | | | | | | |
| 9 | Color of mature | Green | | Green Group 136C, | | 144C to 144B | |
| | leaf upper | 143C and some 61B | | with very little red | | Both surfaces | |
| | surface/lower | More red than | | (mostly Green 136C | | | |
| | surface | ‘Cherokee Brave’ | | for the growing | | | |
| | | and red is persistent | | season) | | | |
| | | through growing | | | | | |
| | | season/ | | | | | |
| | | Green 143C | | | | | |
| 10 | Color of leaves in | Red-Purple 71A | | Red-Purple 71A | | 71C to 71D | |
| | autumn | | | | | | |
| | (observation) | | | | | | |
| 11 | Leaf shape | Ovate | | Ovate | | Ovate | |
| 12 | Leaf Margin | Entire | | Entire | | Entire | |
| 13 | Leaf Tip | Cuspidate | | Cuspidate | | Cuspidate | |
| 14 | Leaf Base | Cuneate | | Cuneate | | Cuneate | |
| 15 | Leaf Venation/ | Palmate/Smooth | | Palmate/Smooth | | Palmate/Smooth | |
| | Texture | with hairs | | with hairs | | with hairs | |
| 16 | Leaf Length | 4.1-6.25 | cm | 5.0-7.2 | cm | 5.0-8.0 | cm |
| 17 | Leaf Width | 0.8-1.2 | cm | 1.0-2.0 | cm | 2.0-2.5 | cm |
| 18 | Petiole Length | <1 | cm | <1 | cm | 1.0-1.3 | cm |
| 19 | Petiole Color | 134C | | 134C | | 149A | |
| 20 | Petiole Texture | Smooth | | Smooth | | Smooth | |
| 21 | Flower diameter | 6.5 mm open | | 6.5 mm open | | 6.5 mm open | |
| | (measurement) | | | | | | |
| 22 | Floret color when | Yellow Green 150A- | | Yellow Green 150A- | | Yellow Green | |
| | open | 150B with some | | 150B with some | | 150A-150B with | |
| | (observation) | purple 76A to 76B | | purple 76A to 76B | | some purple 76A | |
| | on top | on top | | to 76B on top | | | |
| 23 | Uniformity of | See 24-29 | | See 24-29 | | See 24-29 | |
| | bract size | | | | | | |
| | (observation) | | | | | | |
| 24 | Bract overlapping | Overlapping tips | | Slightly overlap | | Very little overlap | |
| | (observation for | and edges | | | | | |
| | both types) | | | | | | |
| 25 | Whole shape of | Spade-shaped with | | Tear-drop with | | Linear | |
| | bracts | point at the base | | blunt tip | | | |
| | (observation) | | | | | | |
| 26 | Inner Bract length | 23.1 | mm | 38.8 | mm | 29.3 | mm |
| | (measurement) | (both types) | | | | | |
| 27 | Inner Bract width | 16.8 | mm | 35.9 | mm | 20.4 | mm |
| | (measurement)- | (both types) | | | | | |
| | modified cleft | | | | | | |
| 28 | Outer Bract | 18.9 | mm | 38.5 | mm | 23.8 | mm |
| | width (measure) | (both types) | | | | | |
| 29 | Outer Bract | 15.3 | mm | 30.3 | mm | 31.8 | mm |
| | length | (both types) | | | | | |
| | (measurement) | | | | | | |
| 30 | Number of bracts | 4 | | 4 | | 4 | |
| 31 | Bract color | 63C-striated red- | | 63C-striated red- | | White 155B | |
| | (light red) | veined, on white | | veined, on white, | | | |
| | | (95%) | | pure white base | | | |
| | | | | of bract | | | |
| 32 | Bract color | 182A to 181B | | 63C striated red- | | Some pink 49D | |
| | (dark red) | (mostly solid color | | veined, on white, | | around margins | |
| | | with some white | | pure white base of | | bleeding towards | |
| | | striation near the | | bract | | center | |
| | | base (<5%)) | | | | | |
| 33 | Cleft in Bract | Yellow Green 145C | | Some are almost | | Reduced and | |
| | | (<5%) | | pure white, others | | Violet purple 93B | |
| | | | | have same color | | to Blush purple | |
| | | | | as the bracts | | group N74C or | |
| | | | | | | creamy white with | |
| | | | | | | purple/red 84D | |
| 34 | Bract duration | Most bracts gone by | | Most bracts gone by | | Most bract gone by | |
| | (both types) | mid-late April | | mid-late April | | mid-late April | |
| 35 | Pedicel Length | 23.8 | mm | 25.2 | mm | 19.5 | mm |
| 36 | Bract variegation | None | | None | | None | |
| | (observation) | | | | | | |
| 37 | Pistil color | Yellow Green | | Yellow Green | | Yellow Green | |
| | (observation) | 150A-150B | | 150A-150B | | 150A-150B | |
| 38 | Fruit shape | Broadly oval | | Broadly oval | | Broadly oval | |
| | (observation) | | | | | | |
| 39 | Fruit length | About 1.5 cm | | About 1.5 cm | | About 1.5 cm | |
| | (measurement) | | | | | | |
| 40 | Fruit color | Red when mature in | | Red when mature in | | Red when mature | |
| | (observation) | fall 45A to 45B | | fall 45A to 45B | | in fall 45A to 45B | |
| 41 | Fragrance | None | | None | | None | |
| | (observation) | | | | | | |
| 42 | Flowering season | Spring | | Spring | | Spring | |
| | (observation) | | | | | | |
| 43 | Flowering time | April | | April | | April | |
| | (observation) | | | | | | |
| 44 | Deciduous or | Deciduous | | Deciduous | | Deciduous | |
| | evergreen | | | | | | |
| | (observation) | | | | | | |
| 45 | Disease resistance | Highly resistant to | | Moderately | | Highly resistant to | |
| | (observation) | powdery mildew | | resistance to | | powdery mildew | |
| | | caused by Erisphe | | powdery mildew | | caused by Erisphe | |
| | | pulchra but some | | caused by Erisphe | | pulchra but some | |
| | | Spot Anthracnose | | pulchra but some | | Spot Anthracnose | |
| | | spotting by Elsinoe | | Spot Anthracnose by | | Elsinoe cornii | |
| | | cornii | | Elsinoe cornii | | | |
| 46 | Bark color | 144A (mottled) | | 144A (mottled) | | 144C | |
| | (mature) | | | | | | |
| 47 | Flower/ | 19.7 | | 25.0 | | 16.4 | |
| | inflorescence | | | | | | |
| | number | | | | | | |
| 48 | Anther color | Purple 86A | | Purple 86A | | Purple 86A | |
| 49 | Flower petal | 3-5 | mm | 3-5 | mm | 3-5 | mm |

| | | | | |
|---|---|---|---|---|
| | length | | | |
| 50 | Flower petal | Purple 76A to 76B | Yellow Green group | Purple 76A to 76B |
| | color (closed) | on top and Yellow | 149B (no purple) | on top and Yellow |
| | | Green 150A-150B | | Green 150A-150B |
| | | near bottom | | near bottom |
| 51 | Flower petal | 150A to 150B | 150A to 150B | 150C |
| | color (open) | | | |

Botanical classification: Cornus florida ‘Erica's Appalachian Sunrise’.

Unique Features: A mixture of two types of bracts, a first one being less than about 5% of the bracts produced that is fully dark red and a second type being more than about 95% of the bracts produced that is similar in color to the bracts produced by the parent ‘Cherokee Brave’. Both types of bracts on ‘Erica's Appalachian Sunset’ are similar in size and significantly smaller than the bracts produced by the parent ‘Cherokee Brave’. Fewer flowers per inflorescence of Erica's Appalachian Sunset’ than on ‘Cherokee Brave’.

Disease susceptibility: ‘Erica's Appalachian Sunrise’ has a strong resistance to Powdery mildew caused by Erisphe pulchra and only some spotting caused by Elsinoe corni, a cosmetic disease with little damage.

Insect damage: None noted.

In the claim:

1. A new and distinct cultivar of Dogwood tree, Cornus Florida, named ‘ERICA'S APPALACHIAN SUNRISE’, as illustrated and described.
